(19) United States
(12) Patent Application Publication
(10) Pub. No.: US 2026/0240900 A1
(43) Pub. Date: Aug. 20, 2026

(54) METHODS OF TREATMENT OF RENAL DISEASES OR DISORDERS MEDIATED BY COMPLEMENT ACTIVITY

(71) Applicant: Renosis, Inc., Southlake, TX (US)

(72) Inventor: Richard Zager, Seattle, WA (US)

(21) Appl. No.: 19/544,443

(22) Filed: Feb. 19, 2026

Related U.S. Application Data
(60) Provisional application No. 63/760,580, filed on Feb. 19, 2025.

(51) Int. Cl. A61K 31/785; A61K 9/00; A61P 13/12

(52) U.S. Cl. CPC A61K 31/785; A61K 9/0053; A61P 13/12

(57) ABSTRACT

Disclosed herein are methods of treating compliment mediated diseases using a crosslinked poly(allyamine) polymer comprising the residues of 2-propen-1-ylamine, or a salt thereof, 1,3-bis(allylamino)propane, or a salt thereof, and 1,2-dichloroethane.

### BACKGROUND

The present disclosure relates to the medical use of a nonabsorbed composition, preferably veverimer, a crosslinked poly(allyamine) polymer comprising the residues of 2-propen-1-ylamine, or a salt thereof, 1,3-bis(allylamino)propane, or a salt thereof, and 1,2-dichloroethane), for the treatment of renal diseases or disorders mediated by complement activity.

In the presence of renal injury, tubule ammonia production per functional nephron increases. Nath K A, Hostetter M K, Hostetter T H. Pathophysiology of chronic tubulointerstitial disease in rats. Interactions of dietary acid load, ammonia, and complement component C3. J Clin Invest. 1985 August; 76 (2): 667-75. doi: 10.1172/JCI112020. PMID: 2993363; PMCID: PMC423874. Although this helps to maintain net acid excretion, it also represents a maladaptive response that can perpetuate renal damage. In part, this is thought to be due to ammonia-C3 binding, thereby generating a C3 convertase, e.g., ref. Nath et al.; see also Brar J E, Quigg R J. Complement activation in the tubulointerstitium: AKI, CKD, and in between. Kidney Int 2014; 86:663-666. The latter triggers downstream activation of the alternative complement cascade, culminating in tubulointerstitial inflammatory cell infiltrates, cytokine pathway activation, endothelin production, and the generation of the C5b-9 membrane attack complex (e.g. Nath et al., Brar et al.).

Anti-glomerular basement membrane disease (anti-GBM disease) is a life-threatening condition characterized by acute renal failure associated with the presence of auto-antibodies with affinity for the glomerular basement membrane (GBM). It is considered a distinct form of vasculitis. Anti-GBM disease is sometimes referred to as Goodpasture disease in literature or, in some cases, Goodpasture syndrome, when the renal disease and lung hemorrhage co-exist.

Several antibody therapies have been developed that target the complement component. These include Eculizumab (Soliris) for the treatment of Paroxysmal Nocturnal Hemoglobinuria (PNH), Atypical Hemolytic Uremic Syndrome (aHUS), Generalized Myasthenia Gravis (gMG), and Neuromyelitis Optica Spectrum Disorder (NMOSD); Ravulizumab (Ultomiris) for the treatment of PNH and aHUS; Crovalimab (PiaSky) for the treatment of PNH; and Pozelimab (Veopoz) for the treatment of Chapele Disease. However, these treatments have generalized effects, and, hence, pose greater side effects, e.g., severe infections.

There is a need for methods of treatment of renal diseases or disorders mediated by complement activity, including specifically anti-GBM disease.

### BRIEF DESCRIPTION OF THE DRAWINGS

FIG. 1 shows spot urinary total protein (uTP)/creatinine and urine albumin (uAlb)/creatinine ratios 1 and 2 weeks after NTS injection with and without veverimer (V) administration.

FIG. 2 shows urinary C5b-9/creatinine ratios in mice 2 weeks post NTS injection, and plasma C3a concentrations at 1- and 2-weeks post NTS injection with and without veverimer (V) administration.

FIG. 3 shows glomerular histology at 2 weeks post NTS injection with and without concomitant veverimer treatment (H and E staining).

FIG. 4 shows NTS-induced cast formation, tubular injury, and interstitial nephritis (H and E staining).

FIG. 5 shows C45 staining of renal tissues from a normal mouse and from NTS injected mice without or with veverimer treatment.

FIG. 6 shows C5b-9 deposition, assessed by immunohistochemistry, at 2 weeks post NTS injection with and without veverimer treatment.

### DETAILED DESCRIPTION

The present inventors have assessed whether veverimer, via its ability to suppress ammoniagenesis, could mitigate complement activation and injury in the NTS nephritis model. Veverimer is an oral, Na+ free, non-absorbed polymer that binds H+ within the gastrointestinal (GI) tract. This stimulates GI carbonic anhydrase-mediated bicarbonate production and systemic bicarbonate uptake.

In one embodiment, the pharmaceutical composition comprises veverimer, a polymeric drug that can have the following structural properties: Veverimer can be poly(allylamine-co-N,N′-diallyl-1,3-diaminopropane-co-1,2-diaminoethane).

More specifically, veverimer can be a poly[(A¹,V³-di(prop-2-en-1-yl)propane-1,3-diamine)-co-prop-2-en-1-amine], crosslinked with A,A-ethane-1,2-diyl bridges (the mole ratio is»2:5:2 (i.e. it is about 2:about 5:about 2 of A¹,A³-di(prop-2-en-1-yl)propane-1,3-diamine:prop-2-en-1-amine: N, A/′-ethane-1,2-diyl bridges)) polymer.

A structural representation of veverimer can be Formula 1:

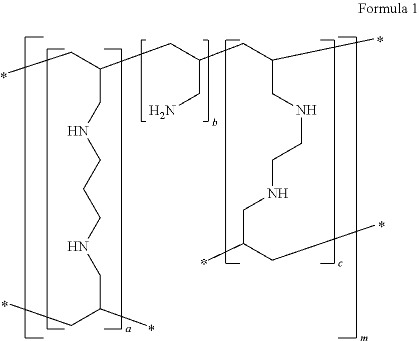

wherein a=residue of A/,/V′-diallyl-1,3-diaminopropane dihydrochloride, b=residue of allylamine, c=residue of 1,2-dichloroethane (ethylene crosslink between two amines); an ethylene linkage between two allylamine groups is shown as an example of one of many possible linkages between amines, m=a large number indicating an extended polymer network.

Veverimer can comprise the following amounts of residues of monomers: a) 20-25 mol % residue of N,N′-diallyl-1,3-diaminopropane, or a salt thereof, (also called 1,3-bis(allylamino)propane, or a salt thereof), b) 50-60 mol % residue of 2-propen-1-ylamine, or a salt thereof, and c) 20-25 mol % residue of 1,2-dichloroethane, wherein the total mol % of the residues is not greater than 100 mol %.

Veverimer can have a carbon to nitrogen weight ratio in the range of about 3.7:1 to about 3.8:1, respectively. The carbon to nitrogen weight ratio may be determined by elemental analysis. For example, the carbon to nitrogen weight ratio may be determined by elemental analysis using a Perkin-Elmer 2400 Elemental Analyzer as more fully described elsewhere herein.

Veverimer can be a nonabsorbable composition that is insoluble, e.g. under physiological conditions.

Veverimer can have a median particle size of greater than 1 micrometer and less than 1 millimeter. The particle size of veverimer may be measured by wet laser diffraction using Mie theory.

Veverimer may be prepared as follows:

Veverimer is obtainable by first copolymerizing 2-propen-1-ylamine, or a salt thereof, and 1,3-bis(allylamino)propane, or a salt thereof, to form a poly(allylamine) polymer, followed by crosslinking the poly(allylamine) polymer with 1,2-dichloroethane.

For example, veverimer is obtainable by first copolymerizing 2-propen-1-ylamine hydrochloride and 1,3-bis(allylamino)propane dihydrochloride to form a poly(allylamine) polymer, followed by crosslinking the poly(allylamine) polymer with 1,2-dichloroethane.

In one embodiment, the pharmaceutical composition comprises a mixture of any of the previously-identified nonabsorbable materials. For example, in one embodiment, the pharmaceutical composition comprises a mixture of a cation exchange composition with at least one anion exchange composition, amphoteric ion exchange composition, or neutral composition having the capacity to bind both protons and anions. In another embodiment, the pharmaceutical composition comprises a mixture of an anion exchange composition with at least one cation exchange composition, amphoteric ion exchange composition, or neutral composition having the capacity to bind both protons and anions. In yet another embodiment, the pharmaceutical composition comprises a mixture of a neutral composition having the capacity to bind both protons and anions with at least one cation exchange composition, amphoteric ion exchange composition, or anion exchange composition.

The present invention may include the use of known analogs of Veverimer that have been described in one or more of the following patent applications: WO 2014/197725—Proton-binding polymers for oral administration; WO 2014/197725—Proton-binding polymers for oral administration; WO/2016/094685—PROTON-BINDING POLYMERS FOR ORAL ADMINISTRATION; WO/2016/094685—PROTON-BINDING POLYMERS FOR ORAL ADMINISTRATION; WO/2017/193050—Compositions for treating acid-based disorders; WO/2017/193064—Compositions for and method of treating acid-based disorders; WO/2017/193024—HCl-binding compositions for and method of treating acid-based disorders; WO/2019/090176—PROTON-BINDING POLYMERS FOR ORAL ADMINISTRATION; WO/2019/090177—PROTON-BINDING POLYMERS FOR ORAL ADMINISTRATION; WO/2019/236636—PROTON-BINDING POLYMERS FOR ORAL ADMINISTRATION; WO/2019/236124—Method of treating Acid-Based Disorders; WO/2019/236639—PROTON-BINDING POLYMERS FOR ORAL ADMINISTRATION; WO/2022/186813—Crosslinked Poly(Allylamine) polymer pharmaceutical compositions; WO/2022/186813—Crosslinked Poly(Allylamine) polymer pharmaceutical compositions; WO/2021/216496—Process for the preparation of 1,3-bis(allylamino)propane; WO/2023/064357—Pharmaceutical Compositions for the Treatment of Lithiasis Disorders; and 62/562,554 US—ONLY-CROSSLINKED POLY(ALLYLAMINE) POLYMER PHARMACEUTICAL COMPOSITIONS.

In one aspect, the present invention involves orally administering a nonabsorbable composition comprising a crosslinked poly(allylamine) polymer, e.g., veverimer, to treat a renal disease or disorder mediated by complement activity. Such diseases or disorders include anti-GBM disease, systemic lupus, membranous nephropathy, IgA nephropathy, membranoproliferative glomerulonephritis, C3 nephropathy, focal segmental glomerulosclerosis, or hemolytic uremic syndrome.

Large increases in proteinuria and albuminura were noted at both 1- and 2-weeks post NTS injection. At both timepoints, veverimer suppressed these urinary protein increases by approximately 50-60%, indicating renal protection. The latter was confirmed by histologic analyses, given that significant reductions in both glomerular and tubulointerstitial injuries were observed. Consistent with these findings, veverimer also suppressed pro-inflammatory (IL-6, MCP-1, ET-1) and pro-fibrotic (TGFß1) cytokine gene expression and induced a 75% decrease in NGAL mRNA, a tubular injury biomarker. Hence, each of the above findings, and a relative preservation of cortical LDH, clearly indicate a marked veverimer-mediated renal protective effect. Noteworthy is that veverimer's beneficial effect could be expressed even when it was administered 24 hrs after NTS injection, a time at which heavy proteinuria was already expressed. This indicates a therapeutic, or renal ‘rescue’, effect.

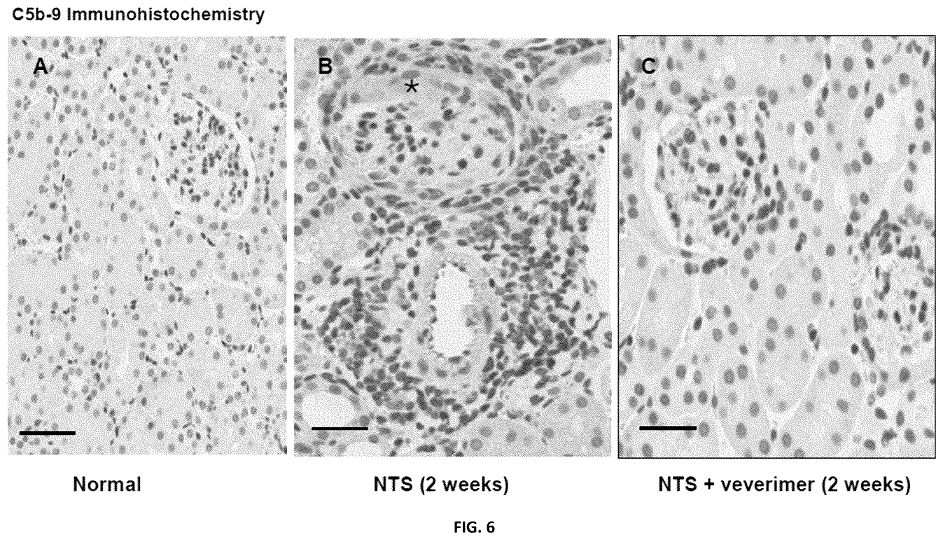

To test the concept that these changes may have been due, at least in part, to suppressed complement activation, tubulointerstitial C5b-9 deposition was probed by immunohistochemistry. As shown in FIG. 6, NTS activated the complement cascade, as evidenced by heavy renal C5b-9 deposition, particularly within the tubulointerstitial space. This change was virtually completely suppressed in veverimer-treated mice. Given that urinary C5b-9 concentrations can serve as a semi-quantitative marker of renal complement activation (e.g., in IgA nephropathy; ref. 25,26), C5b-9 concentrations were measured in 2-week post NTS urine samples. NTS treated mice demonstrated a doubling of urinary C5b-9 concentrations, a response which was blocked by veverimer treatment. To further test for NTS-induced complement activation, plasma levels of the C3 activation fragment, C3a, were measured. That a near doubling of plasma C3a concentrations was observed at both 1- and 2-weeks post NTS injection, and that these increases were completely suppressed by veverimer treatment, support the above C3b-9 immunohistochemistry data. Hence, these findings support the concept that veverimer suppressed activation of the complement cascade. The latter was certainly due to veverimer-mediated bicarbonate generation, given that oral bicarbonate blocked urinary C5b-9 increases and suppressed proteinuria following NTS injection.

Although the above data clearly indicate that veverimer suppressed complement activation, it remains to be proven that the observed renal protection was predominantly due to complement pathway inhibition, per se. In this regard, ammonia generation has been suggested to evoke tissue injury via both complement dependent and independent (31-33) pathways. As examples of the latter, excess ammonia may exert adverse effects on gene expression, apoptosis, immune responsiveness, and metabolic and innate immune signaling pathways (33). Further suggesting that factors in addition to complement activation may have been at play is that complement depletion, induced in rodents by cobra venom factor injection or gene C3 and C4 knockout, have conferred only partial protection against NTS (e.g. ref. 16-20). Hence, while the current studies clearly indicate that veverimer suppressed ammoniagenesis, complement activation, and disease severity, establishing a direct mechanistic link between these results require further study.

Another outstanding issue is that NTS nephritis has two injury phases: the ‘heterologous’ injury phase (induced by sheep anti-GBM antibody injection), followed within ^{~}4-7 days by the autologous injury phase (mediated by mouse anti-sheep antibody production). We cannot state during which (or potentially in both), injury phase veverimer exerts its protective effects. We can state the degree of autologous phase antibody production was not impacted by veverimer treatment, given the results of the mouse anti-sheep antibody titer assessments. Future detailed kinetics studies in which different injury pathways are examined at multiple time points in the presence and absence of veverimer might be helpful in resolving this complex issue.

This study has demonstrated that veverimer administration to mice results in bicarbonate generation, bicarbonaturia, decreased renal ammoniagenesis, suppression of the complement cascade, and reduced severity of NTS-induced nephritis. Veverimer-mediated bicarbonate generation appears to be the proximate cause of these outcomes. However, the operative ‘downstream’ mechanistic links between decreased ammoniagenesis, complement pathway suppression, and NTS disease reduction, and the timing at which this protection is evoked, remain to be more completely defined.

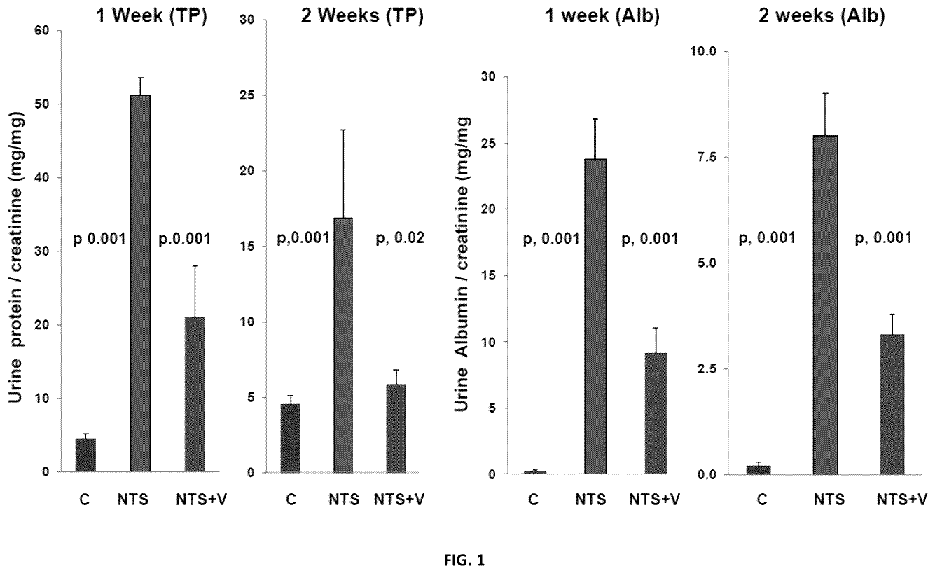

FIG. 1. Spot urinary total protein (uTP)/creatinine and urine albumin (uAlb)/creatinine ratios 1 and 2 weeks after NTS injection with and without veverimer (V) administration. Approximate 10-fold and 100-fold increases in urine protein/creatinine and urine albumin/creatinine ratios, respectively, were observed at 1 week post NTS injection. At the 2-week time point, urinary protein excretion appeared to be decreasing but remained 5-fold (uTP) and 40-fold (uAlb) elevated over normal values. Veverimer induced an approximate 50-75% reduction in these values at both time points, indicating a persistent renal protective effect. (n,6 per assessment, ANOVA with Tukey HSD after test).

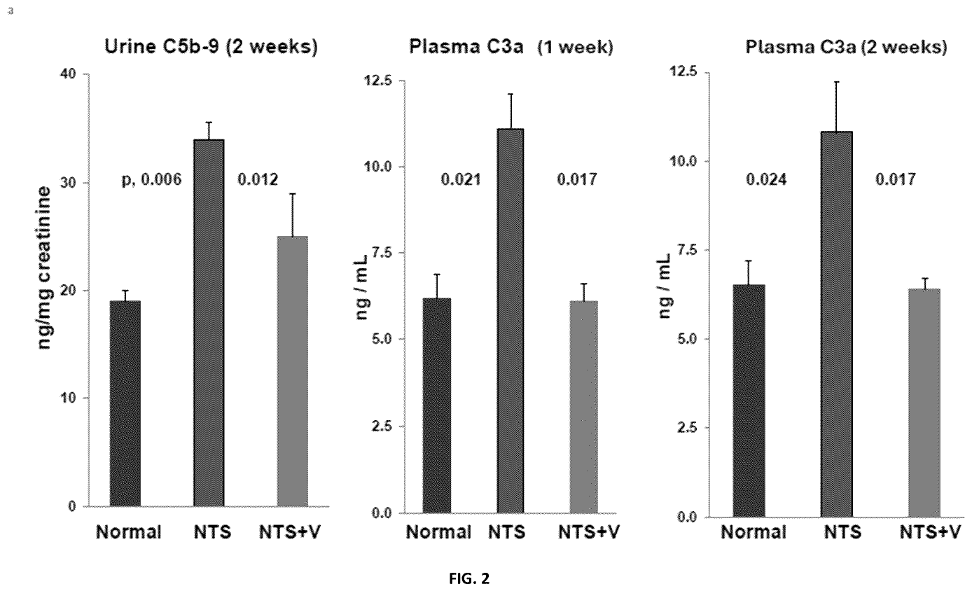

FIG. 2. Urinary C5b-9/creatinine ratios in mice 2 weeks post NTS injection, and plasma C3a concentrations at 1- and 2-weeks post NTS injection with and without veverimer (V) administration. At two weeks post NTS injection, an approximate 80% increase in urine C5b-9/creatinine ratios was observed (compared to baseline values). Further indicating complement activation, NTS caused significant increases in plasma C3a concentrations at both the 1- and 2-week time points. When NTS-injected mice were treated with veverimer, both urinary C5b-9 ratios and plasma C3a levels were normalized, with values not significantly differing from those observed in normal urine samples. (n,6 per assessment, ANOVA with Tukey HSD after test).

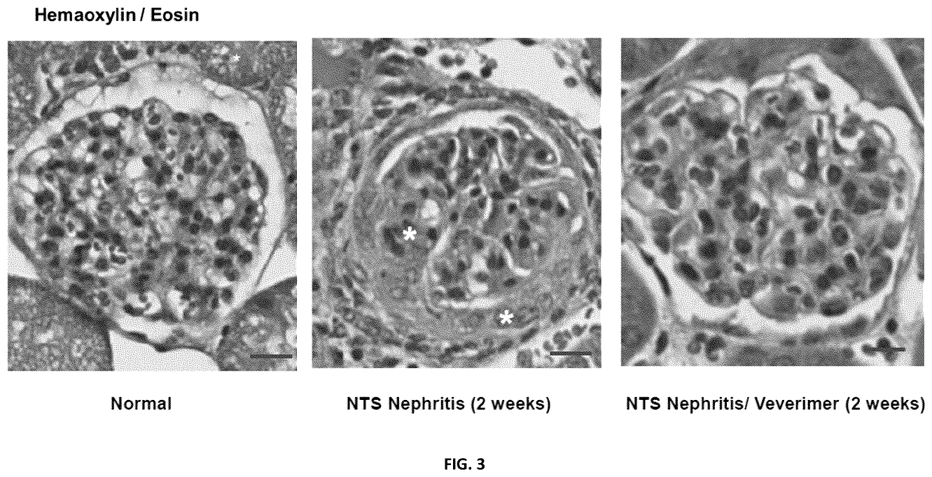

FIG. 3. Glomerular histology at 2 weeks post NTS injection with and without concomitant veverimer treatment (H and E staining). NTS induced acute glomerulonephritis with crescent (*) formation and mesangial expansion. Veverimer significantly reduced glomerular injury, as evidenced by an absence of crescent formation (consistent with a 50-60% decrease in proteinuria as shown in FIG. 1). Scale bars, 50 M.

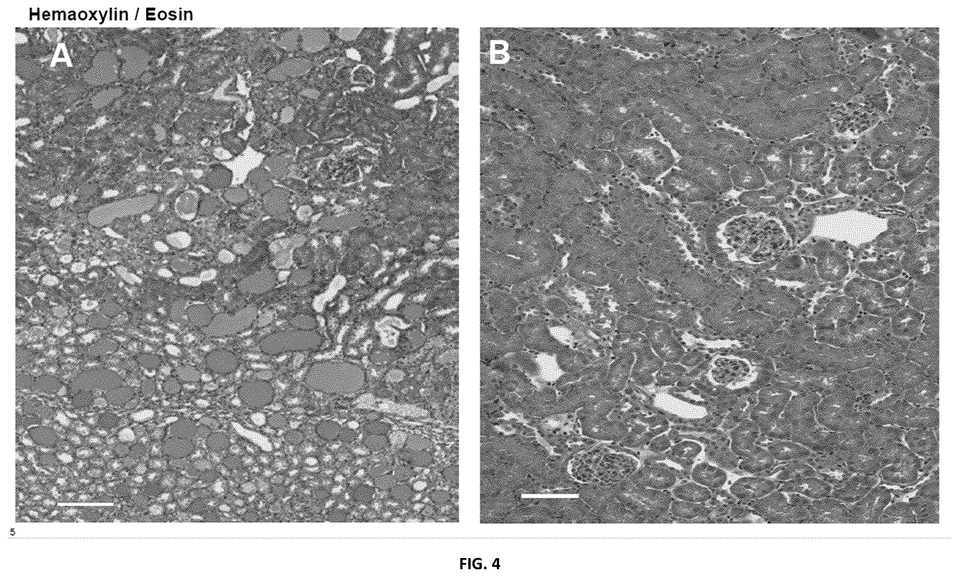

FIG. 4. NTS-induced cast formation, tubular injury, and interstitial nephritis (H and E staining). As shown in the left-hand panel (A), NTS injection resulted in extensive cast formation, tubular degeneration, cellular debris sloughing into tubular lumina (black arrows), and interstitial inflammation. These changes were almost completely prevented by veverimer treatment (panel B). Scale bars, 200 μM.

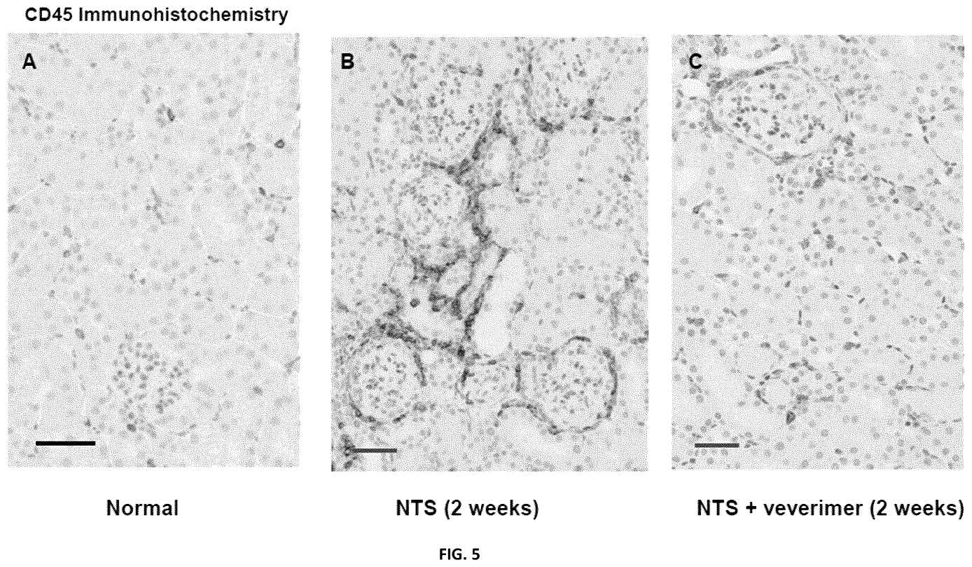

FIG. 5. C45 staining of renal tissues from a normal mouse and from NTS injected mice without or with veverimer treatment. Only occasional CD45+ cells were observed in normal renal tissue (panel A). In contrast, increased tubulointerstitial and peri-glomerular CD45+ cell accumulation were observed at 2 weeks following NTS (B) injection. Veverimer treatment decreased peritubular CD45+ cell accumulation, as depicted in panel C. Scale bars, 100 M.

FIG. 6. C5b-9 deposition, assessed by immunohistochemistry, at 2 weeks post NTS injection with and without veverimer treatment. No evidence of C5b-9 deposition was seen in normal kidney sections (panel A). NTS caused heavy C5b-9 deposition throughout the histologic sections, as denoted by beige/light brown C5b-9 staining. This was accompanied by histologic evidence of tubulointerstitial nephritis with marked inflammatory cell infiltrates. A glomerular crescent is depicted (*). Conversely, C5b-9 deposits were not observed with veverimer treatment (panel B). Scale bars, 85 μM.

TABLE 1
Assessments performed on normal mouse samples ± veverimer treatment.

| | | | Plasma | Urine | |
|---|---|---|---|---|---|
| Group | Blood pH | Urine pH | cHCO3⁻ | NH3/Creat | NHE3 |
| Control | 7.36 ± 0.03 | 5.5 ± 0.1 | 16.5 ± 0.42 | 6.2 ± 0.93 | 186 ± 2.5 |
| Veverimer | 7.41 ± 0.01 | 7.4 ± 0.1 | 18.5 ± 0.66 | 1.3 ± 0.07 | 153 ± 3.9 |
| P | p, 0.18 | p, 0.0001 | p, 0.03 | p, 0.0001 | p, 0.0005 |

Table 1 legend. Blood/urine pH, calculated plasma bicarbonate (cHCO3; mEq/L), ammonia/creatinine ratios (NH3/Creat) and renal cortical NHE3 concentrations, as assessed by ELISA (ng/mg tissue protein extract). Veverimer treatment caused significant increases in urine pH and plasma cHCO3-concentrations. It also evoked an approximate 80% suppression of urinary ammonia/creatinine ratios and decreased renal cortical NHE3 protein levels.

TABLE 2
Plasma and urine analytes at 1 and 2 weeks
post nephrotoxic serum (NTS) injection.

| Analyte | NTS | NTS + veverimer | P value |
|---|---|---|---|
| Urine pH (week 1) | 5.7 ± 0.03 | 7.8 ± 0.25 | 0.0001 |
| Control, 5.7 ± 0.06 | | | |
| Urine pH (week 2) | 5.8 ± 0.09 | 7.4 ± 0.25 | 0.001 |
| Control, 5.7 ± 0.06 | | | |
| Urine NH4/Creat | 4.25 ± 0.95 | 1.48 ± 0.21 | 0.002 |
| Control 6.1 ± 0.5 | | | |
| PcHCO₃ (week 2) | 17.1 ± 0.31 | 17.2 ± 0.46 | NS |
| Control, 18 ± 0.08; | | | |
| mEq/L | | | |
| Blood pH (week 2) | 7.48 ± 0.01 | 7.45 ± 0.01 | NS |
| Control, 7.4 ± 0.03 | | | |
| BUN (week 2) | 27 ± 1 | 23 ± 1 | NS |
| Control, 22 ± 2; mg/dl | | | |
| P Creat (week 2) | 0.3 ± 0.01 | 0.3 ± 0.01 | NS |
| Control, 0.3 ± 0.03 mg/dL | | | |

Table 2 Legend. Urine pH, calculated plasma bicarbonate (P cHCO3-) blood urea nitrogen (BUN, mg/dL) and plasma creatinine (P creat, mg/dL) levels, determined at either 1- or 2-weeks following injection of nephrotoxic serum (NTS)+veverimer treatment. The p values were derived by comparing the NTS vs the NTS+veverimer groups. Baseline urine values were obtained prior to NTS injection. Control P cHO3-, pH, BUN, and plasma creatinine concentrations were determined in 5 normal (non NTS injected) mice.

TABLE 3
Veverimer: Renal tissue analyses of injury mediators and markers,

| | | | NTS + | |
|---|---|---|---|---|
| Analyte | Normal | NTS | veverimer | P value |
| NGAL mRNA | 0.1 ± 0.001 | 3.5 ± 1.1 | 0.8 ± 0.2 | 0.03 |
| MCP-1 mRNA | 0.03 ± 0.01 | 1.43 ± 0.24 | 0.96 ± 0.19 | 0.06 |
| IL-6 mRNA | 0.14 ± 0.01 | 1.94 ± 0.32 | 1.08 ± 0.10 | 0.01 |
| TGFβ1 mRNA | 0.68 ± 0.04 | 1.71 ± 0.16 | 1.27 ± 0.10 | 0.05 |
| ET-1 mRNA | 0.18 ± 0.02 | 0.70 ± 0.11 | 0.40 ± 0.04 | 0.03 |
| LDH units/gm | 1000 ± 12* | 747 ± 10 | 882 ± 20 | 0.01 |

Table 3 legend. Renal cortical mRNA and tissue lactate dehydrogenase (LDH) levels in normal mice and in mice at 2 weeks post NTS injection±veverimer treatment. NTS nephritis caused a significant rise in each of the mRNAs compared to values seen in normal mice (each p<0.001). The p values presented in the right-hand column represent the comparisons of the NTS vs NTS/veverimer treated mice. In each instance, veverimer significantly suppressed the NTS-induced mRNA increases. Statistics by ANOVA/Tukey HSD after test. Veverimer-mediated protection was also indicated by modest, but significant, preservation of renal cortical LDH content (NTS vs NTS+veverimer; unpaired t test). * The control LDH values represent historical controls and are shown as a reference.

TABLE 4
NTS nephritis with and without Na bicarbonate therapy

| | | | NTS + | |
|---|---|---|---|---|
| Analyte | Control | NTS | NaHCO3 | P value |
| U prot/Cr | 4.8 ± 0.6 | 59 ± 11 | 27 ± 3.4 | 0.03 |
| U C5b-9/Cr | 20 ± 0.92 | 38 ± 4.6 | 16 ± 2.1 | <0.001 |
| Arterial pH | 7.4 ± 0.03 | 7.43 ± 0.03 | 7.40 ± 0.01 | NS |
| Urine pH | 5.7 ± 0.06 | 5.7 ± 0.06 | 8.4 ± 0.18 | 0.001 |
| p HCO3⁻ | 18 ± 0.08 | 16 ± 0.26 | 20.0 ± 0.69 | 0.008 |
| BUN | 22 ± 1 | 33 ± 2 | 27 ± 1 | 0.023 |
| P Cr | 0.35 ± 0.01 | 0.35 ± 0.02 | 0.35 ± 0.02 | NS |
| Urine NH3/Cr | 9.0 ± 1.81 | 3.87 ± 0.56 | 0.37 ± 0.05 | <0.001 |

Table 4 legend: Urine and plasma analytes in control mice and mice that were subjected to NTS without or with concomitant Na bicarbonate therapy (assessments performed 1 week post NTS injection). NTS caused large increases in the urine protein/creatinine (Cr) and urine C5b-9/Cr ratios (p<0.01; not depicted). Each of these increases were significantly reduced by bicarbonate therapy. NTS, by itself, decreased urinary NH3/creatinine concentrations compared to values seen in control mice (see text; p<0.01). Bicarbonate further depressed urinary ammonia/creatinine ratios in NTS injected animals. As expected, bicarbonate increased urine pH, and P cHCO3-concentrations. The depicted p values reflect the NTS vs NTS+bicarbonate treatment groups. NTS slightly increased the BUN, but not the plasma creatinine concentrations.

TABLE 5
Mouse anti-sheep IgG antibody levels
at 2 weeks post NTS injection.

| Plasma Dilution | 1:32 | 1:64 | 1:128 | 1:256 |
|---|---|---|---|---|
| NTS, controls | 3.65 ± 0.29 | 3.2 ± 0.13 | 3.0 ± 0.16 | 2.8 ± 0.3 |
| NTS + veverimer | 4.12 ± 0.27 | 4.0 ± 0.28 | 3.6 ± 0.30 | 3.2 ± 0.3 |
| P | 0.25 | 0.046 | 0.64 | 0.20 |

Table 5. Legend. Plasma samples were serially diluted from 1:2 to 1:256. The enhanced chemiluminescence (ECL) antibody signals were converted to log base 10. Starting at serial dilution 1:32, approximate halving of the ECL signals commenced. At all dilutions, the ECL antibody signals were higher in plasma samples from veverimer treated animals. Assay plates were coated with normal sheep IgG (R&D Systems, Cat #5-001-A; 1 ug/mL) i.e., capture antigen. Assay buffer and 1% casein blocker were obtained from Thermo Fisher (cat. #37528). Following solution coating, 25 μl samples of serial plasma dilutions (from 1:2 to 1:256) were added per well. The plates were incubated with shaking (600 rpm)×2 hrs and then washed. Goat anti-mouse IgG (MSD cat. #R32AC-1, labeled with caged ruthenium) was added, followed by washing. A read buffer (MSD) was applied and the plates were read on an MSD (SQ120) instrument. Samples were run in duplicates.

Results: Veverimer increased plasma bicarbonate/urinary pH, reduced urinary ammonia, and decreased NHE3 in normal mice. Veverimer also reduced NTS-induced proteinuria/albuminura, urinary ammonia, and C5b-9 excretion (by ^{~}60%, ^{~}75%, ^{~}50%, respectively). Significant reductions in NTS-induced glomerular/tubulointerstitial injury, inflammatory/profibrotic gene expression, renal C5b-9 deposition, and suppressed plasma C3a levels were observed. Oral bicarbonate also conferred protection, implicating bicarbonate in veverimer's beneficial effect.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

What is claimed is:

1. A method of treating a renal disease or disorder mediated by complement activity wherein the treatment comprises orally administering a nonabsorbable composition comprising a crosslinked poly(allylamine) polymer.

2. The method of claim 1, wherein the nonabsorbable composition is veverimer.

3. The method of claim 1, wherein the complement mediated disorder is anti-GBM disease, systemic lupus, membranous nephropathy, IgA nephropathy, membranoproliferative glomerulonephritis, C3 nephropathy, focal segmental glomerulosclerosis, or hemolytic uremic syndrome.

4. The method of claim 1, wherein the complement mediated disorder is anti-GBM disease.
